(19) United States
(12) Patent Application Publication
(10) Pub. No.: US 2009/0234020 A1
(43) Pub. Date: Sep. 17, 2009

(54) Processes for the preparation of odesmethylvenlafaxine, free from its dimer impurities

(76) Inventors: Valarie Niddam-Hildesheim, Kadima (IL); Tamar Nidam, Yehud (IL); Yuri Vollerner, Lod (IL); Miri Adler, Petach Tikva (IL); Miri Kravitz, Rehovot (IL); Sharona Shachan-tov, Kfar-Saba (IL)

Correspondence Address: KENYON & KENYON LLP, ONE BROADWAY, NEW YORK, NY, 10004, US

(21) Appl. No.: 12/381,080

(22) Filed: Mar. 6, 2009

Related U.S. Application Data
(60) Provisional application No. 61/034,372, filed on Mar. 6, 2008.

(51) Int. Cl. A61K 31/137; C07C 211/00; G01N 33/00; A61P 25/24

(52) U.S. Cl. 514/649; 564/305; 436/112

(57) ABSTRACT

The present invention provides isolated O-desmethylvenlafaxine impurities ODV-Dimer and ODV-N-Dimer, their use as a reference marker and reference standard, and a process for the preparation of O-desmethylvenlafaxine free from said impurities

### CROSS REFERENCE TO RELATED APPLICATIONS

The present invention claims the benefit of the following U.S. Provisional Patent Application No. 61/034,372, filed Mar. 6, 2008. The contents of this application is incorporated herein by reference.

### FIELD OF THE INVENTION

The invention encompasses isolated 4-[2-(dimethylamino)-1-(1-hydroxycyclohexyl)ethyl]-2-({5-[2-(dimethylamino)-1-(1-hydroxycyclohexyl) ethyl]-2-hydroxyphenyl}methyl) phenol, and isolated 4-[2-(dimethylamino)-1-(1-hydroxycyclohexyl)ethyl]-2-({[2-(1-hydroxycyclohexyl)-2-(4-hydroxyphenyl) ethyl](methyl)amino}methyl) phenol O-desmethylvenlafaxine impurities, as well as their use as a reference marker and reference standard, and a process for the preparation of O-desmethylvenlafaxine free from said impurities.

### BACKGROUND OF THE INVENTION

Venlafaxine, (±)-1-[2-(Dimethylamino)-1-(4-methoxyphenyl) ethyl]cyclohexanol is the first of a class of anti-depressants. Venlafaxine acts by inhibiting re-uptake of norepinephrine and serotonin, and is an alternative to the tricyclic anti-depressants and selective re-uptake inhibitors. Venlafaxine has the following chemical formula, Formula I:

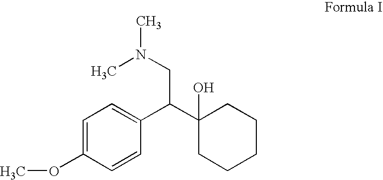

O-desmethylvenlafaxine, 4-[2-(dimethylamino)-1-(1-hydroxycyclohexyl)ethyl]phenol, is reported to be a metabolite of venlafaxine and has been reported to inhibit norepinephrine and serotonin uptake. See Klamerus, K. J. et al., “Introduction of the Composite Parameter to the Pharmacokinetics of Venlafaxine and its Active O-Desmethyl Metabolite,” J. Clin. Pharmacol. 32:716-724 (1992). O-desmethylvenlafaxine has the following chemical formula, Formula II:

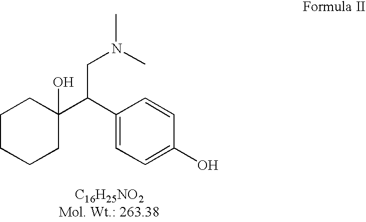

Processes for the synthesis of O-desmethylvenlafaxine, comprising a step of demethylation of the methoxy group of venlafaxine, are described in U.S. Pat. Nos. 7,026,508 and 6,689,912, and in U.S. publication No. 2005/0197392.

The synthesis disclosed in the above references is performed according to the following scheme:

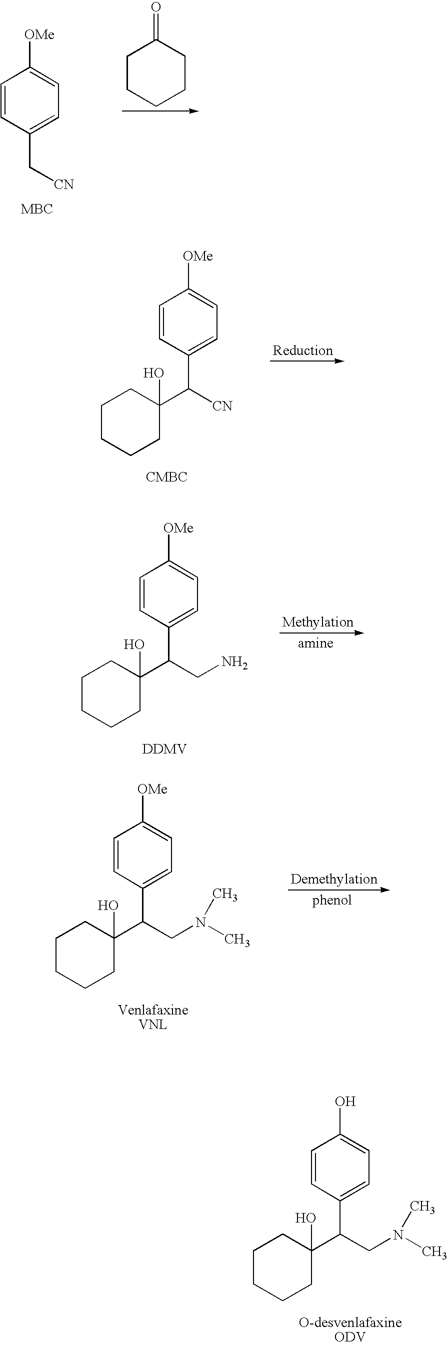

Wherein “MBC” refers to methyl benzyl cyanide, “CMBC” refers to cyclohexyl methylbenzyl cyanide, “DDMV” refers to didesmethyl venlafaxine, and “ODV” refers to O-desmethylvenlafaxine.

Like any synthetic compound, O-desmethylvenlafaxine can contain extraneous compounds or impurities. These impurities may be, for example, starting materials, by-products of the reaction, products of side reactions, or degradation products. Impurities in O-desmethylvenlafaxine, or any active pharmaceutical ingredient (“API”), are undesirable and, in extreme cases, might even be harmful to a patient being treated with a dosage form containing the API.

The purity of an API produced in a manufacturing process is critical for commercialization. The U.S. Food and Drug Administration (“FDA”) requires that process impurities be maintained below set limits. For example, in its ICH Q7A guidance for API manufacturers, the FDA specifies the quality of raw materials that may be used, as well as acceptable process conditions, such as temperature, pressure, time, and stoichiometric ratios, including purification steps, such as crystallization, distillation, and liquid-liquid extraction. See ICH Good Manufacturing Practice Guide for Active Pharmaceutical Ingredients, Q7A, Current Step 4 Version (Nov. 10, 2000).

The product of a chemical reaction is rarely a single compound with sufficient purity to comply with pharmaceutical standards. Side products and by-products of the reaction and adjunct reagents used in the reaction will, in most cases, also be present in the product. At certain stages during processing of an API, such as O-desmethylvenlafaxine, it must be analyzed for purity, typically, by high performance liquid chromatography (“HPLC”) or thin-layer chromatography (“TLC”), to determine if it is suitable for continued processing and, ultimately, for use in a pharmaceutical product. The FDA requires that an API is as free of impurities as possible, so that it is as safe as possible for clinical use. For example, the FDA recommends that the amounts of some impurities be limited to less than 0.1 percent. See ICH Good Manufacturing Practice Guide for Active Pharmaceutical Ingredients, Q7A, Current Step 4 Version (Nov. 10, 2000).

Generally, side products, by-products, and adjunct reagents (collectively “impurities”) are identified spectroscopically and/or with another physical method, and then associated with a peak position, such as that in a chromatogram, or a spot on a TLC plate. See Strobel, H. A., et al., CHEMICAL INSTRUMENTATION: A SYSTEMATIC APPROACH, 953, 3d ed. (Wiley & Sons, New York 1989). Once a particular impurity has been associated with a peak position, the impurity can be identified in a sample by its relative position in the chromatogram, where the position in the chromatogram is measured in minutes between injection of the sample on the column and elution of the impurity through the detector. The relative position in the chromatogram is known as the “retention time.”

The retention time can vary about a mean value based upon the condition of the instrumentation, as well as many other factors. To mitigate the effects such variations have upon accurate identification of an impurity, practitioners often use “relative retention time” (“RRT”) to identify impurities. See supra Strobel at 922. The RRT of an impurity is calculated by dividing the retention time of the impurity by the retention time of a reference marker. The reference marker may be the API in which the impurity is present, or may be another compound that is either present in or added to the sample. A reference marker should be present in the sample in an amount that is sufficiently large to be detectable, but not in an amount large enough to saturate the column.

Those skilled in the art of drug manufacturing research and development understand that a relatively pure compound can be used as a “reference standard.” A reference standard is similar to a reference marker, except that it may be used not only to identify the impurity, but also to quantify the amount of the impurity present in the sample.

A reference standard is an “external standard,” when a solution of a known concentration of the reference standard and an unknown mixture are analyzed separately using the same technique. See supra Strobel at 924; Snyder, L. R., et al., INTRODUCTION TO MODERN LIQUID CHROMATOGRAPHY, 549, 2d ed. (John Wiley & Sons, New York 1979). The amount of the impurity in the sample can be determined by comparing the magnitude of the detector response for the reference standard to that for the impurity. See U.S. Pat. No. 6,333,198, hereby incorporated by reference.

The reference standard can also be used as an “internal standard,” i.e., one that is directly added to the sample in a predetermined amount. When the reference standard is an internal standard, a “response factor,” which compensates for differences in the sensitivity of the detector to the impurity and the reference standard, is used to quantify the amount of the impurity in the sample. See supra Strobel at 894. For this purpose, the reference standard is added directly to the mixture, and is known as an “internal standard.” See supra Strobel at 925; Snyder at 552.

The technique of “standard addition” can also be used to quantify the amount of the impurity. This technique is used where the sample contains an unknown detectable amount of the reference standard. In a “standard addition,” at least two samples are prepared by adding known and differing amounts of the internal standard. See supra Strobel at 391-393; Snyder at 571-572. The proportion of the detector response due to the reference standard present in the sample can be determined by plotting the detector response against the amount of the reference standard added to each of the samples, and extrapolating the plot to zero. See supra Strobel at 392, FIG. 11.4.

There is, therefore, a need in the art to detect, isolate, and remove said impurities from samples of O-desmethylvenlafaxine.

### SUMMARY OF THE INVENTION

In one embodiment, the invention encompasses isolated 4-[2-(dimethylamino)-1-(1-hydroxycyclohexyl)ethyl]-2-({5-[2-(dimethylamino)-1-(1-hydroxycyclohexyl)ethyl]-2-hydroxyphenyl}methyl)phenol (“ODV-Dimer”) having the formula:

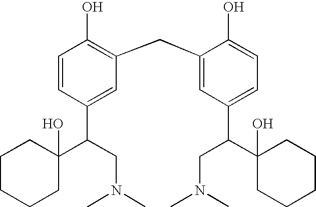

In one embodiment, the invention encompasses isolated 4-[2-(dimethylamino)-1-(1-hydroxycyclohexyl)ethyl]-2-({[2-(1-hydroxycyclohexyl)-2-(4-hydroxyphenyl)ethyl](methyl)amino}methyl)phenol (“ODV-N-Dimer”) having the formula:

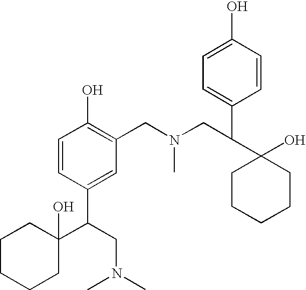

In one embodiment, the present invention encompasses a method for qualitatively analyzing the purity of O-desmethylvenlafaxine or salts thereof comprising:

a) providing a reference sample comprising O-desmethylvenlafaxine or salts thereof and ODV-Dimer or ODV-N-Dimer or a combination thereof;
b) analyzing the reference sample by HPLC and determining the relative retention time of the ODV-Dimer and/or the ODV-N-Dimer compared to O-desmethylvenlafaxine or salts thereof;
c) analyzing a sample of O-desmethylvenlafaxine or salts thereof by HPLC and determining the relative retention times of the contents of the sample as compared to O-desmethylvenlafaxine or salts thereof; and
d) comparing the relative retention times calculated in step c) to the relative retention time calculated in step b) for the ODV-Dimer and/or the ODV-N-Dimer, wherein if any of the relative retention times calculated in step c) corresponds with the relative retention time of the ODV-Dimer or the ODV-N-Dimer, ODV-Dimer and/or ODV-N-Dimer are present in the sample of O-desmethylvenlafaxine or salts thereof.

In one embodiment, the present invention encompasses a method for determining the amount of ODV-Dimer and/or ODV-N-Dimer in a sample of O-desmethylvenlafaxine or salts thereof sample comprising:

a) measuring by HPLC the area under the peak corresponding to the ODV-Dimer and/or the ODV-N-Dimer in a sample of O-desmethylvenlafaxine or salts thereof having an unknown amount of the ODV-Dimer or the ODV-N-Dimer or a combination thereof;
b) measuring by HPLC the area under a peak corresponding to the ODV-Dimer and/or the ODV-N-Dimer in a reference standard comprising a known amount of the ODV-Dimer and/or the ODV-N-Dimer; and
c) determining the amount of the ODV-Dimer and/or the ODV-N-Dimer in the sample of O-desmethylvenlafaxine or salts thereof by comparing the area calculated in step a) to the area calculated in step b).

In one embodiment, the present invention encompasses a method for determining the amount of ODV-Dimer and/or ODV-N-Dimer in a sample of O-desmethylvenlafaxine or salts thereof using ODV-Dimer or ODV-N-Dimer comprising:

a) measuring by HPLC the area under the peak corresponding to the ODV-Dimer and/or the ODV-N-Dimer in a sample of O-desmethylvenlafaxine or salts thereof having an unknown amount of the ODV-Dimer and/or the ODV-N-Dimer;
b) measuring by HPLC the area under a peak corresponding to O-desmethylvenlafaxine or salts thereof in a reference standard having a known amount of O-desmethylvenlafaxine or salts thereof;
c) determining a response factor for the HPLC area under the peak by comparing the area calculated in step b) with the known amount of O-desmethylvenlafaxine in the standard reference; and
d) determining the amount of the ODV-Dimer and/or the ODV-N-Dimer in the sample of O-desmethylvenlafaxine or salts thereof by comparing the area calculated in step a) with the response factor calculated in step c).

### DETAILED DESCRIPTION OF THE INVENTION

O-desmethylvenlafaxine may contain the impurities 4-[2-(dimethylamino)-1-(1-hydroxycyclohexyl)ethyl]-2-({5-[2-(dimethylamino)-1-(1-hydroxycyclohexyl)ethyl]-2-hydroxyphenyl}methyl)phenol (“ODV-Dimer”), and 4-[2-(dimethylamino)-1-(1-hydroxycyclohexyl)ethyl]-2-({[2-(1-hydroxycyclohexyl)-2-(4-hydroxyphenyl)ethyl](methyl)amino}methyl)phenol (“ODV-N-Dimer”).

The invention provides methods for detecting and isolating the O-desmethylvenlafaxine impurities ODV-Dimer and ODV-N-Dimer, as well as methods for removing those impurities from samples of O-desmethylvenlafaxine and salts thereof.

As used herein, the term “room temperature” refers to a temperature of about 20° C. to about 35° C., more preferably about 25° C. to about 35° C., more preferably about 25° C. to about 30° C., and most preferably about 25° C.

As used herein, the term “reference standard” refers to a compound that may be used both for quantitative and qualitative analysis of an active pharmaceutical ingredient. For example, the HPLC retention time of the reference standard allows a relative retention time with respect to the active pharmaceutical ingredient to be determined, thus making qualitative analysis possible. Furthermore, the concentration of the compound in solution before injection into an HPLC column allows the areas under the HPLC peaks to be compared, thus making quantitative analysis possible.

A “reference marker” is used in qualitative analysis to identify components of a mixture based upon their position, e.g., in a chromatogram or on a Thin Layer Chromatography (TLC) plate (Strobel pp. 921, 922, 953). For this purpose, the compound does not necessarily have to be added to the mixture if it is present in the mixture. A “reference marker” is used only for qualitative analysis, while a reference standard may be used for quantitative or qualitative analysis, or both. Hence, a reference marker is a subset of a reference standard, and is included within the definition of a reference standard.

As used herein, “isolated” in reference to the ODV-Dimer or the ODV-N-Dimer impurity that is physically separated from the reaction mixture. For example, the separation can be done by elution from a HPLC column and further drying the impurity.

The invention encompasses isolated 4-[2-(dimethylamino)-1-(1-hydroxycyclohexyl)ethyl]-2-({5-[2-(dimethylamino)-1-(1-hydroxycyclohexyl) ethyl]-2-hydroxyphenyl}methyl)phenol (“ODV-Dimer”), which is an impurity in O-desmethylvenlafaxine. The ODV-Dimer is represented by the following chemical structure:

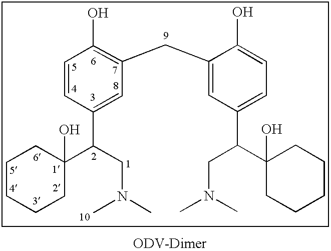

The ODV-Dimer may be characterized by at least one of: a ¹H NMR (300 MHz, DMSO-d6) spectrum: δ 6.81 (dd, 2H, J=8.1 Hz and 1.5 Hz, H₄), 6.72 (d, 2H, J=1.5 Hz, H₈), 6.67 (d, 2H, J=8.1 Hz, H₅), 3.72 (d, 2H, J=16 Hz, H_{9α}), 3.67 (d, 2H, J=16 Hz, H_{9β}), 3.00 (dd, 2H, J=12 Hz and 9 Hz, H_{1α}), 2.28 (dd, 2H, J=16 Hz and 6 Hz, H_{1β}), 2.62 (dd, 2H, J=9 Hz and 6 Hz, H₂), 2.13 (s, 9H, H₁₀), 0.8-1.6 (m, 10H, H_{2′-6′}); a ¹³C NMR (DMSO-d6) spectrum δ 181.1 (C-3), 153.26 (C-6), 131.14 (C-8), 127.11 (C-4), 125.69 (C-7), 113.92 (C-5), 72.56 (C-1′), 60.31 (C-1), 51.67 (C-2), 45 (C-10), 37.05 (C-2′), 31.78 (C-6′), 29.45 (C-9), 25.63 (C-4′), 21.15 (C-5′), 21.08 (C-3′); and a MS Fab+ (MH⁺=539.4).

The invention further encompasses isolated 4-[2-(dimethylamino)-1-(1-hydroxycyclohexyl)ethyl]-2-({[2-(1-hydroxycyclohexyl)-2-(4-hydroxyphenyl)ethyl](methyl)amino}methyl)phenol (“ODV-N-Dimer”), which is an impurity in O-desmethylvenlafaxine. The ODV-N-Dimer is represented by the following chemical structure:

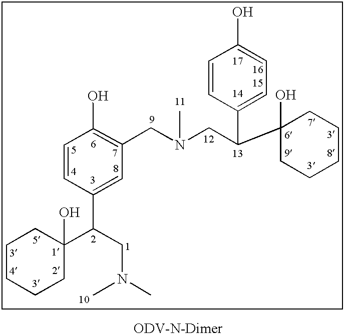

The ODV-N-Dimer may be characterized by at least one of: a ¹H NMR (300 MHz, CD₃OD) spectrum: δ 7.02 (d, 2H, J=8.5, H₁₅), 6.72 (d, 2H, J=8.4, H₁₆), 6.59 (dd, 1H, J=8.5 Hz and 2 Hz, H₄), 6.58 (d, 1H, J=8.5 Hz, H₅), 6.85 (d, 1H, J=2 Hz, H₈), 3.64 (d, 1H, J=13 Hz, H_{9α}), 3.57 (d, 1H, J=13 Hz, H_{9β}), 3.16 (dd, 1H, J=13 Hz and 9 Hz, H_{1α}), 3.04 (dd, 1H, J=12 Hz and 6 Hz, H_{12α}), 2.98 (dd, 1H, J=12 Hz and 9.5 Hz, H_{12β}), 2.85 (dd, 1H, J=9.5 Hz and 6 Hz, H₁₃), 2.79 (dd, 1H, J=9 Hz and 6 Hz, H₂), 2.5 (dd, 1H, J=13 Hz and 6 Hz, Hp) 2.25 (s, 6H, H₁₀), 2.18 (s, 3H, H₁₁), 0.9-1.7 (m, 10H, H_{2′-5′,7′-9′}); a ¹³C NMR (CD₃OD) spectrum 6161.48 (C-15), 157.17 (C-6, C-17), 132.25 (C-14), 132.21 (C-3), 131.48 (C-4, C-8), 123.6 (C-7), 115.9 (C-16), 75.61 (C-1′), 74.64 (C-6′), 61.68 (C-1), 61.48 (C-9), 58.75 (C-12), 54.02 (C-13), 53.58 (C-2), 45.75 (C-10), 41.78 (C-11), 38.35 (C-9′), 37.81 (C-2′), 34.38 (C-7′), 33.2 (C-5′), 26.99 (C-8′), 26.94 (C-4′), 22.79-22.6 (C-3′); and a MS ES+ (MH⁺=525).

The invention further encompasses compositions comprising either the ODV-Dimer or the ODV-N-Dimer mentioned above, wherein the amount of O-desmethylvenlafaxine is less than about 0.2% by area HPLC. Preferably less than about 0.15% area by HPLC, preferably less than about 0.1% area by HPLC, for example, between about 0.03% to about 0.15% or between about 0.07% and 0.1%.

The invention also encompasses a process for preparing ODV-Dimer comprising eluting an ODV sample, containing the ODV-Dimer in a column, silica gel column chromatography (230-400 mesh), using CH₂Cl₂, MeOH, and NH₄OH as eluent solvents. Preferably, the eluent solvents ratio is CH₂Cl₂:MeOH:NH₄OH 19:1:0.2.

The invention also encompasses a process for preparing ODV-N-Dimer comprising eluting an ODV sample, containing the ODV-N-Dimer, from a silica gel (230-400 mesh) column chromatography, using CH₂Cl₂, and MeOH as eluent solvents. Preferably, the eluent solvents ratio is CH₂Cl₂:MeOH 95:5. Preferably, the ODV-N-Dimer is purified by chromatography on a combiflash.

Optionally, prior to the process described above, the ODV sample, containing ODV-N-Dimer can be transferred through a column, to avoid unwanted substances, using CH₂Cl₂, MeOH, and H₂O as eluent solvents. Preferably, the eluent solvents ration is CH₂Cl₂:MeOH:H₂O 65:35:8.

Also provided is a process for preparing O-desmethylvenlafaxine having less than about 0.2% area by HPLC of any of the impurities ODV-Dimer or ODV-N-Dimer comprising: a) demethylating didesmethyl venlafaxine to obtain tridesmethylvenlafaxine; b) reductive amination of tridesmethylvenlafaxine to obtain O-desmethylvenlafaxine; c) slurring O-desmethylvenlafaxine in a C₁-C₄ alcohol solvent at the reflux temperature of the solvent; and d) cooling the slurry to a temperature of about 0° C. to room temperature, for sufficient time and slurrying at this temperature before filtration to obtain O-desmethylvenlafaxine having less than about 0.2% area by HPLC of any of the impurities ODV-Dimer or ODV-N-Dimer. Preferably, the obtained O-desmethylvenlafaxine has less than about 0.15% area by HPLC, preferably less than about 0.1% area by HPLC, for example, between about 0.03% to about 0.15% or between about 0.07% and 0.1%, of any of the impurities ODV-Dimer or ODV-N-Dimer. Preferably a sufficient time is about 2 hours to about 8 hours. Preferably the C₁-C₄ alcohols is isopropanol.

Preferably, the process described above results in O-desmethylvenlafaxine having less than about 0.2% area by HPLC of combined ODV-Dimer and ODV-N-Dimer, preferably less than about 0.15% area by HPLC, preferably less than about 0.1% area by HPLC, for example, between about 0.03% to about 0.15% or between about 0.07% and 0.1%.

The demethylation of disdesmethylvenlafaxine in step a) may be carried out, for example, as described in co-pending application U.S. Ser. No. 11/881,731, the contents of which are incorporated herein by reference. Preferably, didesmethyl venlafaxine is reacted with a sulfide containing demethylating agent at an elevated temperature in the presence of high boiling point solvent.

The high boiling point solvent may be selected from the group consisting of: toluene, dimethylformamide (“DMF”), dimethylsulfoxide (“DMSO”), N-methyl-2-pyridone, N-methyl-2-pyrrolidone (NMP), 1-methyl-2-pyrrolidinone, dimethylacetamide (“DMA”), polyethylene glycol, Marlotherm, silicon oil, N,N′-dimethylpropyleneurea (“DMPU”), dimethylolethyleneurea (“DMEU”), Hexamethylphosphoramide (“HMPA”), diethylformamide (“DEF”), diethyleneamine (“DEA”), morpholine, sulfolane, phenylether and mixtures thereof. More preferably, the high boiling point solvent is polyethylene glycol, NMP or DMA.

The sulfide containing demethylating agent may be selected from metal sulfides, having either a valence of −1 or −2, thiolates and thiols. Preferably, the demethylating agent is a mercaptan or a salt thereof, a salt of a thioalcohol, or sodium sulfide. A preferred thiolate is a high molecular weight thiolate or arene thiolate. More preferably, the sulfide containing demethylating agent is sodium dodecanethiolate or thiophenol. The sodium dodecanethiolate can be obtained by any method known to the skilled artisan, such as combining sodium methoxide, methanol and dodecanethiol.

As used herein, the term “elevated temperature” means a temperature greater than about 50° C., but less than a temperature at which about 10% or more of either the reactants or the product degrades over the course of the reaction.

The reductive amination of tridesmethylvenlafaxine in step b) may be carried out, for example, as described in co-pending application U.S. Ser. No. 12/001,070, the contents of which are incorporated herein by reference. Preferably, a solution of tridesmethyl venlafaxine and a formaldehyde source (such as a reaction mixture) is provided, and O-desmethylvenlafaxine is recovered from the reaction.

In order to yield an even purer product, the O-desmethylvenlafaxine having less than about 0.2% area by HPLC of any of the impurities ODV-Dimer or ODV-N-Dimer is further re-crystallized from C₁-C₄ alcohols. Preferably the C₁-C₄ alcohol is isopropanol.

Preferably, the O-desmethylvenlafaxine obtained after re-crystallization has less than about 0.15% area by HPLC of any of the impurities ODV-Dimer or ODV-N-Dimer, more preferably, less than about 0.1%, most preferably, less than about 0.05%.

Also provided is a process for preparing O-desmethylvenlafaxine succinate having less than about 0.2% area by HPLC of any of the impurities ODV-Dimer or ODV-N-Dimer comprising re-crystallizing O-desmethyl venlafaxine having less than about 0.2% area by HPLC of any of the impurities ODV-Dimer or ODV-N-Dimer from a solution of a C₁-C₄ alcohol, water and succinic acid. Preferably, the O-desmethylvenlafaxine and/or the O-desmethylvenlafaxine succinate have less than about 0.15% area by HPLC of any of the impurities ODV-Dimer or ODV-N-Dimer, preferably less than about 0.1% area by HPLC. Preferably the C₁-C₄ alcohol is isopropanol.

The ODV-Dimer and/or the ODV-N-Dimer are useful as reference markers for O-desmethylvenlafaxine or salts thereof. As such, they may be used in order to detect the presence of the ODV-Dimer and/or the ODV-N-Dimer in a sample of O-desmethylvenlafaxine or salts thereof.

The invention encompasses the use of the ODV-Dimer and/or the ODV-N-Dimer as reference markers to qualitatively analyze the purity of O-desmethylvenlafaxine or salts thereof. The method comprises: a) providing a reference sample comprising O-desmethylvenlafaxine or salts thereof and the ODV-Dimer or the ODV-N-Dimer or a combination thereof; b) analyzing the reference sample by HPLC and determining the relative retention time of the ODV-Dimer and/or the ODV-N-Dimer compared to O-desmethylvenlafaxine or salts thereof; c) analyzing a sample of O-desmethylvenlafaxine or salts thereof by HPLC and determining the relative retention times of the contents of the sample as compared to O-desmethylvenlafaxine or salts thereof; and d) comparing the relative retention times calculated in step c) to the relative retention time calculated in step b) for the ODV-Dimer and/or the ODV-N-Dimer, wherein if any of the relative retention times calculated in step c) correspond with the relative retention time of the ODV-Dimer or the ODV-N-Dimer, the ODV-Dimer and/or the ODV-N-Dimer are present in the sample of O-desmethylvenlafaxine or salts thereof.

The ODV-Dimer and/or the ODV-N-Dimer are also useful as reference standards for O-desmethylvenlafaxine or salts thereof. As such, they may be used in order to quantify the amount of the ODV-Dimer and/or the ODV-N-Dimer in a sample of O-desmethylvenlafaxine or salts thereof.

The ODV-Dimer and/or the ODV-N-Dimer may be used as an external reference standard for O-desmethylvenlafaxine or salts thereof. The use of the ODV-Dimer and/or the ODV-N-Dimer as reference standards for determining the amount of the ODV-Dimer or the ODV-N-Dimer or a combination thereof in a O-desmethylvenlafaxine or salts thereof sample comprising: a) measuring by HPLC the area under the peak corresponding to the ODV-Dimer and/or the ODV-N-Dimer in a sample of O-desmethylvenlafaxine or salts thereof having an unknown amount of the ODV-Dimer or the ODV-N-Dimer or a combination thereof; b) measuring by HPLC the area under a peak corresponding to the ODV-Dimer and/or the ODV-N-Dimer in a reference standard comprising a known amount of the ODV-Dimer or the ODV-N-Dimer or a combination thereof; and c) determining the amount of the ODV-Dimer or the ODV-N-Dimer or a combination thereof in the O-desmethylvenlafaxine or salts thereof sample by comparing the area calculated in step a) to the area calculated in step b).

The invention further encompasses a quantification method for determining the amount of the ODV-Dimer or the ODV-N-Dimer or a combination thereof in an O-desmethylvenlafaxine or salts thereof sample using ODV-Dimer and/or ODV-N-Dimer. The method comprises: a) measuring by HPLC the area under the peak corresponding to the ODV-Dimer and/or the ODV-N-Dimer in a sample of O-desmethylvenlafaxine or salts thereof having an unknown amount of the ODV-Dimer and/or the ODV-N-Dimer;

b) measuring by HPLC the area under a peak corresponding to O-desmethylvenlafaxine or salts thereof in a reference standard having a known amount of O-desmethylvenlafaxine or salts thereof;
c) determining a response factor for the HPLC area under the peak by comparing the area calculated in step b) with the known amount of O-desmethylvenlafaxine in the standard reference; and
d) determining the amount of the ODV-Dimer and/or the ODV-N-Dimer in the sample of O-desmethylvenlafaxine or salts thereof by comparing the area calculated in step a) with the response factor calculated in step c). The response factor may be calculated by dividing the known concentration in the reference standard, for example the known O-desmethylvenlafaxine concentration, with the area under the curve by HPLC determined for the same compound, for example O-desmethylvenlafaxine, in the reference standard.

Having described the invention with reference to certain preferred embodiments, other embodiments will become apparent to one skilled in the art from consideration of the specification. The invention is further defined by reference to the following examples describing in detail the preparation and analysis of O-desmethylvenlafaxine or salts thereof. It will be apparent to those skilled in the art that many modifications, both to materials and methods, may be practiced without departing from the scope of the invention.

### EXAMPLES

### Nuclear Magnetic Resonance (“NMR”) Spectroscopy

NMR spectroscopy was performed on a Bruker DPX (300 MHz) using DMSO-d6 or CD₂OD as solvents.

### High Performance Liquid Chromatography (“HPLC”)

A high performance liquid chromatograph with a Zorbax SB Phenyl 75×4.6 3.5μ40° C. column and an ultraviolet detector at 230 nm was used with detection limit of 0.03% and quantitative limit of 0.05%. The flow rate was 1.5 ml/minute.

The mobile phase was comprised of two eluents (A and B). Eluent A was 70% (0.005M Dodecyl Sulphate+0.07% TEA(triethylamine) pH-3.0 with H₂SO₄) and 30% acetonitrile. Eluent B was 40% (0.005M Dodecyl Sulphate+0.07% TEA (triethylamine) pH-3.0 with H₂SO₄) and 60% acetonitrile.

Samples of O-desmethylvenlafaxine were dissolved in a 1:1 (volume:volume) mixture of water and MeOH. Each sample contained about 0.5 mg O-desmethylvenlafaxine per milliliter of solvent. The samples were carried through the column by gradient elution under the following conditions: 3 minutes of 100% eluent A, followed by an increase in eluent B from 0 to 60% from 3 to 35 minutes.

### Example 1

### Preparation of TDMV (Tridesmethylvenlafaxine) from DDMV

To a 1 liter reactor equipped with mechanical stirrer, condenser, dean-stark and thermometer were added at room temperature under flow of nitrogen DDMV.HCl (100 g 0.35 mol), 62% Na₂S hydrate (48.5 g, 0.386 mol) and NMP (200 ml).

The reaction mixture was heated to 50° C. and kept at this temperature for 0.5 hours, the temperature was raised to 185° C. in a period of 3 h, and then the reaction mixture was kept at this temperature until completion of the reaction (5-6 h) The mixture was cooled to 90° C. A solution of water (500 ml) and then a solution of succinic acid (17 g, 0.14 mol.) in water (500 ml) were added dropwise at this temperature in order to reach pH 10-11.

The obtained slurry was cooled to 10° C. during 5 hours and stirred at this temperature overnight. The solid was filtered under reduced pressure washed with water (3×100 ml). The solid was dried overnight in a vacuum oven at 50° C. to obtain 76.24 g of TDMV (yield=91%, HPLC purity 98.53%).

### Example 2

### Preparation of ODV Base Crude from TDMV

To a 2 liter reactor equipped with mechanical stirrer, condenser and thermometer were added at room temperature under flow of nitrogen TDMV (70 g, 0.29 mol.), paraformaldehyde (44.6 g, 1.49 mmol), NaOH (23.8 g, 0.595 mmol) and IPA (1000 ml). Formic acid (137.0 g 2.98 mmol) was added dropwise. The reaction mixture was heated to reflux and kept in reflux for 9 hours. Water (350 ml) was added and the pH was adjusted to 9-9.5 using a 47% NaOH (96 gr).

The obtained slurry was cooled to 5° C. and stirred at this temperature for overnight. The solid was filtered under reduced pressure, washed with H₂O (3×70 ml) and dried overnight at 50° C. under vacuum to obtain solid 64.57 g of ODV base crude (yield=81.7%, HPLC purity 97.6% ODV-Dimer 0.07%, ODV-N-Dimer 1.66%).

### Example 3

### Preparation of ODV Base Pure

To a one liter reactor equipped with mechanical stirrer, condenser and thermometer were added at room temperature ODV base crude (60 g, 0.22 mol) and IPA (900 ml). The mixture was heated to reflux (83° C.) and kept at this temperature for 1 hour. The suspension was then cooled to 25° C. during 5 hours and stirred at this temperature overnight.

The solid was filtered under reduced pressure and washed with EPA (2×60 ml). The solid was dried overnight in a vacuum oven at 50° C. to obtain 52.7 g of ODV base pure (yield=89.82%, HPLC purity 99.85%, ODV-Dimer—not detected, ODV-N-Dimer 0.1%).

### Example 4

### Preparation of ODV Base Cryst

To 2 liter reactor equipped with mechanical stirrer, condenser and thermometer were added at room temperature ODV base pure (50 g, 0.19 mol) and IPA (1500 ml). The mixture was heated to clear solution (83° C.) hot filtration was done and kept at this temperature for 1 hour. The solution was then cooled to 0° C. during 5 hours and stirred at this temperature overnight.

The solid was filtered under reduced pressure and washed with IPA (2×50 ml). The solid was dried overnight in a vacuum oven at 50° C. to obtain 45.2 g of ODV base cryst (yield=91%, HPLC purity 99.95% ODV-Dimer not detected, ODV-N-Dimer 0.03%).

### Example 5

### Preparation of ODV Succinate

To 0.5 liter reactor equipped with mechanical stirrer, condenser and thermometer were added at room temperature ODV base pure (50 g, 0.1 g mol) and IPA (250 ml). The mixture was heated to reflux (83° C.).

A solution of water (100 ml) and succinic acid (24.65 g, 0.21 mol.) was added at this temperature and kept at this temperature for 1 hr. The mixture was then cooled to 25° C. during 5 hours and stirred at this temperature overnight. The solid was filtered under reduced pressure and washed with IPA (2×50 ml). The solid was dried overnight in a vacuum oven at 50° C. to obtain 64.1 gr of ODV succinate (yield=90%, HPLC purity 99.89%, ODV-Dimer not detected, ODV-N-Dimer 0.08%).

### Example 6

### Preparation of ODV-N-Dimer

To a 0.5 liter reactor equipped with mechanical stirrer, condenser and thermometer were added, at room temperature, TDMV (62.2 g, 0.264 mol.), formaldehyde 24% (100 g, 0.8 mol) and MeOH (100 ml). The reaction mixture was heated to 75° C. and kept at this temperature for 3 days, the suspension was then cooled to 25° C. pH was adjusted to 8.5 with 47% NaOH. The solid was filtered under reduced pressure and washed with H₂O (3×60 ml).

The filtrate with 24.8% ODV-N-dimer was basified to pH 12 and extracted with EtOAc. After evaporation of the solvent, the reaction crude 13.6 g was purified by column chromatography (500 g silica gel, diameter 7 cm, eluent CH₂Cl₂/MeOH/H₂O 65/35/8). Elution of 1.5 L in erlemeyer and then the fractions were collected in tubes of 50 ml. In the fraction 10, after evaporation was collected 2 g of a mixture containing 27.3% ODV-N-dimer.

This fraction was again purified by chromatography on a combiflash (120 g column CH₂Cl₂/MeOH 95/5) to get 0.26 g ODV N-dimer with a purity of 80% (HPLC area). A new column chromatography was performed (eluent CH₂Cl₂/MeOH/H₂O 65/35/8) in order to get 70 mg of ODV N-dimer having an HPLC purity of 94.3%.

N—O-ODV Dimer (in CD₃OD)

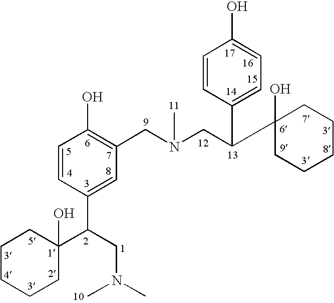

| | C | H | | |
|---|---|---|---|---|
| | Chem. Shift, | Chem. Shift, | | |
| No | ppm | ppm | Multiplicity | J, Hz |
| 1a | 61.68 | 3.16 | dd | 13, 9 |
| 1b | | 2.5 | dd | 13, 6 |
| 2 | 53.58 | 2.79 | dd | 9, 6 |
| 3 | 132.21 | — | — | — |
| 4 | 131.48 | 6.59 | dd | 8.5, 2 |
| 5 | 116.12 | 6.58 | d | 8.5 |
| 6 | 157.17 | — | — | — |
| 7 | 123.6 | — | — | — |
| 8 | 131.48 | 6.85 | d | 2 |
| 9a | 61.48 | 3.57 | d | 13 |
| 9b | | 3.64 | d | 13 |
| 10 | 45.75 | 2.25 | s | — |
| 11 | 41.78 | 2.18 | s | — |
| 12a | 58.75 | 3.04 | dd | 12, 6 |
| 12b | | 2.98 | dd | 12, 9.5 |
| 13 | 54.02 | 2.85 | dd | 9.5, 6 |
| 14 | 132.25 | — | — | — |
| 15 | 161.48 | 7.02 | | |
| 16 | 115.9 | 6.72 | | |
| 17 | 157.17 | — | — | — |
| 1′ | 75.61 | — | — | — |
| 2′ | 37.81 | 0.9-1.7 | m | — |
| 3′ | 22.6-22.79 | | | |
| 4′ | 26.94 | | | |
| 5′ | 33.2 | | | |
| 6′ | 74.64 | — | — | — |
| 7′ | 34.39 | 0.9-1.7 | m | — |
| 8′ | 26.99 | | | |
| 9′ | 38.35 | | | |

### Example 7

### Preparation of ODV-Dimer

The separation was done by column chromatography on silica gel (7 cm diameter). ODV crude 10 gr (containing 0.7% dimer and 98% ODV according to HPLC) was charged on the column. The column was gradually eluted with eluent of: CH₂Cl₂:MeOH:NH₄OH 19:1:0.2.

After all the ODV was eluted, the ODV dimer started to elute at fractions 45-53 to give 100 mg of the desired product (98% purity).

The column was monitored by TLC, TLC conditions were: CH₂Cl₂: MeOH 9:1 and 4 drops NH₄OH.

ODV dimer R_{f}=0.25

ODV R_{f}=0.68

ODV-Dimer (in DMSO-d6)

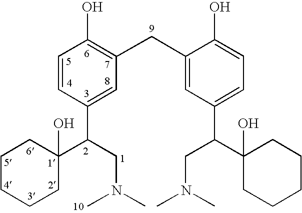

| | C | H | | |
|---|---|---|---|---|
| | Chem. Shift, | Chem. Shift, | | |
| No | ppm | ppm | Multiplicity | J, Hz |
| 1a | 60.31 | 3 | dd | 12, 9 |
| 1b | | 2.28 | dd | 12, 6 |
| 2 | 51.67 | 2.62 | dd | 9, 6 |
| 3 | 181.1 | — | — | — |
| 4 | 127.11 | 6.81 | dd | 8.1, 1.5 |
| 5 | 113.92 | 6.67 | d | 8.1 |
| 6 | 153.26 | — | — | — |
| 7 | 125.69 | — | — | — |
| 8 | 131.14 | 6.72 | d | 1.5 |
| 9a | 29.45 | 3.72 | d | 16 |
| 9b | | 3.67 | d | 16 |
| 10 | 45 | 2.13 | s | — |
| 1′ | 72.56 | — | — | — |
| 2′ | 37.05 | 0.8-1.6 | m | — |
| 3′ | 21.08 | | | |
| 4′ | 25.63 | | | |
| 5′ | 21.15 | | | |
| 6′ | 31.78 | | | |

1. Isolated 4-[2-(dimethylamino)-1-(1-hydroxycyclohexyl)ethyl]-2-({5-[2-(dimethylamino)-1-(1-hydroxycyclohexyl)ethyl]-2-hydroxyphenyl}methyl)phenol (“ODV-Dimer”) having the formula:

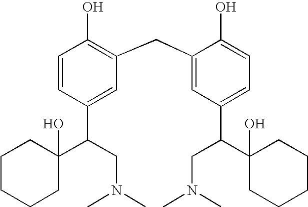

2. The isolated 4-(2-(dimethylamino)-1-(1-hydroxycyclohexyl)ethyl)-2-(5-(2-(dimethylamino)-1-(1-hydroxycyclohexyl)ethyl)-2-hydroxybenzyl)phenol (“ODV-Dimer”) of claim 1, characterized by ¹H NMR (in DMSO-d6) spectrum with signals at about δ 6.81, 6.72, 6.67, 3.72, 3.67, 3.00, 2.28, 2.62, 2.13, 0.8-1.6; a ¹³C NMR (in DMSO-d6) δ 181.1 (C-3), 153.26 (C-6), 131.14 (C-8), 127.11 (C-4), 125.69 (C-7), 113.92 (C-5), 72.56 (C-1′), 60.31 (C-1), 51.67 (C-2), 45 (C-10), 37.05 (C-2′), 31.78 (C-6′), 29.45 (C-9), 25.63 (C-4′), 21.15 (C-5′), 21.08 (C-3′); and a combinations thereof. MS Fab+ (MH⁺=539.4).

3. Isolated 4-[2-(dimethylamino)-1-(1-hydroxycyclohexyl)ethyl]-2-({[2-(1-hydroxycyclohexyl)-2-(4-hydroxyphenyl)ethyl](methyl)amino}methyl)phenol (“ODV-N-Dimer”) having the formula:

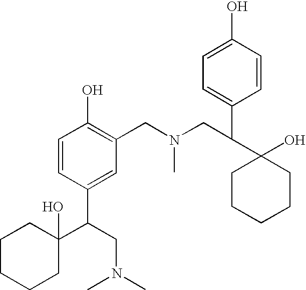

4. The isolated 4-[2-(dimethylamino)-1-(1-hydroxycyclohexyl)ethyl]-2-({[2-(1-hydroxycyclohexyl)-2-(4-hydroxyphenyl)ethyl](methyl)amino}methyl)phenol (“ODV-N-Dimer”) of claim 3, characterized by ¹H NMR (in CD₃OD) spectrum with signals at about δ 7.02, 6.72, 6.59, 6.58, 6.85, 3.64, 3.57, 3.16, 3.04, 2.98, 2.85, 2.79, 2.5, 2.25, 2.18, 0.9-1.7; a ¹³C NMR (in CD₃OD) δ 161.48 (C-15), 157.17 (C-6, C-17), 132.25 (C-14), 132.21 (C-3), 131.48 (C-4, C-8), 123.6 (C-7), 115.9 (C-16), 75.61 (C-1′), 74.64 (C-6′), 61.68 (C-1), 61.48 (C-9), 58.75 (C-12), 54.02 (C-13), 53.58 (C-2), 45.75 (C-10), 41.78 (C-1), 38.35 (C-9′), 37.81 (C-2′), 34.38 (C-7′), 33.2 (C-5′), 26.99 (C-8′), 26.94 (C-4′), 22.79-22.6 (C-3′); a Mass Spectrum of MS ES+ (MH⁺=525); and a combination thereof.

5. A composition comprising the 4-[2-(dimethylamino)-1-(1-hydroxycyclohexyl)ethyl]-2-({5-[2-(dimethylamino)-1-(1-hydroxycyclohexyl)ethyl]-2-hydroxyphenyl}methyl)phenol (“ODV-Dimer”) of claim 1, wherein the amount of O-desmethylvenlafaxine is less than about 0.2% by area HPLC.

6. A composition comprising the 4-[2-(dimethylamino)-1-(1-hydroxycyclohexyl)ethyl]-2-({[2-(1-hydroxycyclohexyl)-2-(4-hydroxyphenyl)ethyl](methyl)amino}methyl)phenol (“ODV-N-Dimer”) of claim 3, wherein the amount of O-desmethylvenlafaxine is less than about 0.2% by area HPLC.

7. A method for qualitatively analyzing the purity level of O-desmethylvenlafaxine or salts thereof comprising:
a) providing a reference sample comprising O-desmethylvenlafaxine or salts thereof and the ODV-Dimer and/or the ODV-N-Dimer;
b) analyzing the reference sample by HPLC and determining the relative retention time of the ODV-Dimer and/or the ODV-N-Dimer compared to O-desmethylvenlafaxine or salts thereof;
c) analyzing a sample of O-desmethylvenlafaxine or salts thereof by HPLC and determining the relative retention times of the contents of the sample as compared to O-desmethylvenlafaxine or salts thereof; and
d) comparing the relative retention times calculated in step c) to the relative retention time calculated in step b) for the ODV-Dimer and/or the ODV-N-Dimer, wherein if any of the relative retention times calculated in step c) are substantially the same as the relative retention time of the ODV-Dimer and/or the ODV-N-Dimer, the ODV-Dimer and/or the ODV-N-Dimer are present in the sample of O-desmethylvenlafaxine or salts thereof.

8. A method for determining the amount of ODV-Dimer and/or ODV-N-Dimer in a sample of O-desmethylvenlafaxine or salts thereof sample comprising:
a) measuring by HPLC the area under the peak corresponding to the ODV-Dimer and/or the ODV-N-Dimer in a sample of O-desmethylvenlafaxine or salts thereof having an unknown amount of the ODV-Dimer and/or the ODV-N-Dimer;
b) measuring by HPLC the area under the peak corresponding to the ODV-Dimer and/or the ODV-N-Dimer in a reference standard comprising a known amount of the ODV-Dimer and/or the ODV-N-Dimer; and
c) determining the amount of the ODV-Dimer and/or the ODV-N-Dimer in the sample of O-desmethylvenlafaxine or salts thereof sample by comparing the area calculated in step a) to the area calculated in step b).

9. A method for determining the amount of the ODV-Dimer and/or the ODV-N-Dimer in O-desmethylvenlafaxine or salts thereof using ODV-Dimer or ODV-N-Dimer comprising:
a) measuring by HPLC the area under the peak corresponding to the ODV-Dimer and/or the ODV-N-Dimer in a sample of O-desmethylvenlafaxine or salts thereof having an unknown amount of the ODV-Dimer and/or the ODV-N-Dimer;
b) measuring by HPLC the area under a peak corresponding to O-desmethylvenlafaxine or salts thereof in a reference standard having a known amount of O-desmethylvenlafaxine or salts thereof;
c) determining a response factor for the HPLC area under the peak by comparing the area calculated in step b) with the known amount of O-desmethylvenlafaxine in the standard reference; and
d) determining the amount of the ODV-Dimer and/or the ODV-N-Dimer in the sample of O-desmethylvenlafaxine or salts thereof by comparing the area calculated in step a) with the response factor calculated in step c).
